(19) United States
(12) Patent Application Publication
(10) Pub. No.: US 2010/0330238 A1
(43) Pub. Date: Dec. 30, 2010

(54) ADHESIVE

(75) Inventors: Matthias Wintermantel, Bergisch Gladback (DE); Christos Karafilidis, Leverkusen (DE); Heinz-Werner Lucas, Bergisch Gladbach (DE); Axel Schmidt, Singapore (SG)

Correspondence Address: CONNOLLY BOVE LODGE & HUTZ, LLP, P O BOX 2207, WILMINGTON, DE, 19899, US

(73) Assignee: Bayer MaterialScience AG, Leverkusen (DE)

(21) Appl. No.: 12/867,692

(22) PCT Filed: Feb. 6, 2009
(86) PCT No.: PCT/EP09/00817

§ 371 Date: Aug. 13, 2010

(30) Foreign Application Priority Data
Feb. 15, 2008 (DE) 10 2008 009 408.0

(51) Int. Cl. C07C 265/00; A23B 4/10

(52) U.S. Cl. 426/125; 560/330

(57) ABSTRACT

The invention relates to the use of special isocyanate-terminated polyurethane prepolymers in adhesive formulations. Said adhesive formulations can be used in applications in which it is important to prevent or minimize migration of adhesive components when the adhesive layer is in direct or indirect contact with substrates that are sensitive thereto.

The present invention relates to the use of special isocyanate-terminated polyurethane prepolymers in adhesive formulations. These adhesive formulations may be used in applications in which it is important to avoid or minimise migrates in direct or indirect contact of the adhesive layer with substrates which are sensitive thereto.

Sensitive substrates within the meaning of the present invention may be, for example, human skin or composite films. The latter are widely used to produce packaging for goods of all kinds. Since it is not possible for all requirements, such as transparency/opacity, printability, barrier properties, sealability and mechanical properties, to be covered by monofilms, co-extruded multi-layer films or extrusion-laminated film composites, composite films in which the individual layers are bonded together using adhesive make up the largest share of the market and thus have immense commercial importance.

The production of food packaging from composite films is of particular significance. Since, on the side facing the food, some of the layers used have low barrier properties against the adhesive components employed, particular attention must be paid to any migration of adhesive components into the food.

In the area of flexible composite packaging films, aromatic polyurethane systems are predominantly used. The migration of aromatic polyisocyanates or their reaction products with water into the food is therefore particularly critical. With water, which is contained in almost all foods, polyisocyanates react with the release of carbon dioxide to form primary aromatic amines. Since primary aromatic amines are toxic, the legislators have issued limits for migrates from food packaging, which it is imperative to observe. For this reason, the adhesives used for the production of composite films must be fully reacted at the time of packing the foods to the extent that migration is safely below the limits. The same applies to the use of such systems on human skin.

Some flexible packaging is sealed after being filled with the food and is then sterilised to kill all germs and to increase the shelf life of the food. Sterilisation is usually performed at temperatures of over 100° C. At these temperatures, aromatic isocyanates can be released from the polyurethane adhesives by recleavage and migrate into the food. For this reason, adhesive formulations based on aliphatic isocyanates are used for the production of flexible film composites according to section 177.1390 FDA. Aliphatic polyisocyanates do not naturally form any primary aromatic amines on reaction with water and are therefore very advantageous for the production of flexible film composites which are intended to undergo sterilisation with the food. As is generally known from polyurethane chemistry, however, aliphatic polyisocyanates have significantly lower reactivity towards polyols than aromatic polyisocyanates. The curing times of aliphatic adhesive formulations at room temperature are therefore extremely long, which means that a long curing, and thus storage, period of the film composite is needed before this is used. If the film composite is subjected to the packing and sterilisation process before the cure is complete, this can lead to delamination of the film composite and thus to the destruction of the packaging owing to incompletely developed interlayer adhesion. Attempts are being made, for both economic and logistic reasons, to minimise the storage time necessary to achieve complete cure. To this end, two different concepts are being employed:
1) Acceleration of the chemical curing reaction of the adhesive formulation by the addition of catalysts.
2) Conditioning the composite films immediately after lamination for 3-7 days at temperatures of at least 40° C. and above.

Thus, for example, WO 2006/026670 describes the use of a polyurethane prepolymer based on aliphatic polyisocyanates in an adhesive formulation which displays adequate interlayer adhesion at 60° C. in three days. In addition to the increased curing temperature, a catalyst (dibutyltin dilaurate, DBTL) is added to the polyurethane prepolymer. Disadvantages are on the one hand the very high curing temperature of 60° C., which requires expensive temperature cabinets or ovens and can lead to roll telescoping and creasing, and on the other hand the catalyst used, which in this case even contains heavy metal.

US-A 2006/0078741 describes the use of catalysts to reduce the curing time of adhesive formulations for the production of film composites. The shorter curing time correlates to the storage time of the film composite before it is used to pack foods. It is a disadvantage of both formulations that the catalyst remains capable of migration within the film composite and can, in principle, contaminate the packed food.

The object of the present invention was therefore to develop adhesive formulations based on an aliphatic polyisocyanate, which are free from catalysts capable of migration and yet can be used at room temperature so that within no more than three days for example adequate interlayer adhesion in composite films is achieved and/or they can be used in the production of wound closure and wound care means. An adequate interlayer adhesion for composite films is 3 N/15 mm or higher.

Surprisingly, it has now been found that adhesive formulations based on aliphatic polyisocyanates develop adequate interlayer adhesion within 3 days at room temperature and yet do not contain any catalyst capable of migration if aliphatic NCO prepolymers are used which contain polymer-bound tertiary amino groups.

The present invention therefore first provides prepolymers based on aliphatic isocyanates containing tertiary amino groups bound to the prepolymer.

In one embodiment of the invention these tertiary amino groups are introduced into the prepolymer by the polyisocyanate component.

In another embodiment of the invention, these tertiary amino groups are introduced into the prepolymer by the isocyanate-reactive component.

Aliphatic polyisocyanates used to produce the prepolymers according to the invention preferably have an NCO content of 11-51 wt. % and a nominal average functionality of 2 to 3.8.

The invention also provides preparations which contain the prepolymers described above.

These preparations are preferably adhesives. These may be used in general for the bonding of substrates; in a preferred embodiment the adhesives are used for the bonding of packaging materials of all kinds and in a particularly preferred form for the production of film composites.

These film composites may be adhesive joints of films or films bonded over their entire surface, as is the case e.g. in composite films.

In particular, food packages produced or sealed with the aid of adhesives produced on the basis of the prepolymers according to the invention are also provided by the present invention. These are preferably composite films with which the food is at least partly covered for the purpose of packing the same. “Partly covered” includes e.g. objects introduced into thermoformed plastics packaging trays if these trays are sealed with a film of this type, optionally also using adhesives according to the invention.

The prepolymers according to the invention can also be used in the production of adhesive and plaster systems for wound closure and care, however, since the absence of residual monomers and the suitability for use at room temperature play an important role here, as does freedom from components capable of migration.

In a preferred embodiment of the present invention, an adhesive formulation for composite materials developing adequate interlayer adhesion within 3 days at room temperature is provided, containing:
A) An isocyanate group-containing component, containing at least one polyisocyanate, characterised in that
   a1) the polyisocyanate
      i. has an average functionality in the range of 2 to 3.8, preferably 2 to 3.2, particularly preferably 2 to 2.4,
      ii. has an NCO content in the range of 11 to 51 wt. %, preferably 21 to 51 wt. %, particularly preferably 23 to 51 wt. %,
   a2) the isocyanate group-containing component contains polymer-bound tertiary amino groups;
B) A polyol component, containing at least one polyhydroxy compound, characterised in that
   a1) the average functionality is in the range from 2 to 4, preferably in the range from 2 to 3.6.
   a2) the OH number is in the range from 6 to 720 mg KOH/g, preferably in the range from 28 to 480 mg KOH/g and particularly preferably in the range from 40 to 240 mg KOH/g;
C) optionally other additives;
   and a process for the production of composite materials using the aforementioned adhesive formulation.

In another preferred use, these or similar adhesive preparations according to the invention are used as surgical adhesives for wound closure and care or in the production of adhesive and plaster systems for wound closure and care, as known e.g. from EP-A 0 897 406 as plasters, or without a textile support directly as a wound adhesive or wound closure means. In addition, active ingredients having a positive effect on wound behaviour may be incorporated into these adhesive preparations. These include, for example, agents having an antimicrobial action, such as antimycotics, and substances having an antibacterial action (antibiotics), corticosteroids, chitosan, dexpanthenol and chlorhexidine gluconate.

The present invention therefore relates to the use of aliphatic isocyanate-terminated polyurethane prepolymers containing amino groups in adhesive formulations for the production of composite films which exhibit adequate interlayer adhesion within a few days at room temperature and are free from catalysts capable of migration, and in the production of medical wound care systems.

The production of the polyisocyanate prepolymers used in the production of A) is known per se to the person skilled in the art and takes place by reacting the polyhydroxy compounds with excess amounts of polyisocyanates. In principle, it is possible to use as the polyisocyanate all organic aliphatic, cycloaliphatic, aromatic or heterocyclic polyisocyanates with at least two isocyanate groups per molecule which are known to the person skilled in the art, as well as mixtures thereof. Examples of suitable aliphatic or cycloaliphatic polyisocyanates are di- or triisocyanates, such as e.g. butane diisocyanate, pentane diisocyanate, hexane diisocyanate (hexamethylene diisocyanate, HDI), 4-isocyanatomethyl-1,8-octane diisocyanate (triisocyanatononane, TIN), or cyclic systems, such as e.g. 4,4′-methylenebis(cyclohexyl isocyanate), 3,5,5-trimethyl-1-isocyanato-3-isocyanatomethylcyclohexane (isophorone diisocyanate, IPDI), as well as ω.ω′-diisocyanato-1,3-dimethylcyclohexane (H₆XDI). As aromatic polyisocyanates it is possible to use e.g. 1,5-naphthalene diisocyanate, diisocyanatodiphenylmethane (2,2′-, 2,4′- and 4,4′-MDI or mixtures thereof), diisocyanatomethylbenzene (2,4- and 2,6-toluene-diisocyanate, TDI), particularly the 2,4- and the 2,6-isomers and technical mixtures of the two isomers, and 1,3-bis(isocyanatomethyl)benzene (XDI). However, the use of aliphatic diisocyanates is preferred, particularly preferably hexane diisocyanate (hexamethylene diisocyanate, HDI), 3,5,5-trimethyl-1-iso cyanato-3-isocyanatomethylcyclohexane (isophorone diisocyanate, IPDI), and 1,3-bis(isocyanatomethyl)benzene (XDI).

In addition, however, it is also possible to use the derivatives, which are known per se, of the aforementioned organic aliphatic, cycloaliphatic or heterocyclic polyisocyanates with a uretdione, allophanate, biuret and/or isocyanurate structure.

As polyhydroxy compounds it is possible to use all compounds known to the person skilled in the art which have an average OH functionality of at least 1.5. These can be, for example, low molecular weight diols (e.g. 1,2-ethanediol, 1,3- or 1,2-propanediol, 1,4-butanediol), triols (e.g. glycerol, trimethylolpropane) and tetraols (e.g. pentaerythritol), but also higher molecular weight polyhydroxy compounds such as polyether polyols, polyester polyols, polycarbonate polyols and polythioether polyols. These polyether polyols preferably have OH numbers of 5 to 620 mg KOH/g, preferably 14 to 550 mg KOH/g and particularly preferably 28 to 480 mg KOH/g. These polyether polyols can be obtained by a method that is known per se by alkoxylation of suitable starter molecules with base catalysis or using double metal cyanide compounds (DMC compounds). Suitable starter molecules for the production of polyether polyols are molecules with at least 2 element-hydrogen bonds that are reactive towards epoxides or any mixtures of such starter molecules. Preferred are polyether polyol mixtures which contain at least one polyol with at least one tertiary amino group. Such tertiary amino group-containing polyether polyols can be produced by alkoxylation of starter molecules or mixtures of starter molecules, containing at least one starter molecule with at least 2 element-hydrogen bonds that are reactive towards epoxides, of which at least one is an NH bond, or low molecular weight polyol compounds containing tertiary amino groups. Examples of suitable starter molecules are ammonia, methylamine, ethylamine, n-propylamine, iso-propylamine, ethanolamine, diethanolamine, triethanolamine, ethylenediamine, ethylenenetriamine, triethanolamine, N-methyldiethanolamine, N,N′-dimethylethylenediamine, tetramethylenediamine, hexamethylenediamine, 2,4-toluenediamine, 2,6-toluenediamine, aniline, diphenylmethane-2,2′-diamine, diphenylmethane-2,4′-diamine, diphenylmethane-4,4′-diamine, 1-aminomethyl-3-amino-1,5,5-trimethylcyclohexane (isophorone diamine), dicyclohexylmethane-4,4′-diamine, xylylenediamine and polyoxyalkyleneamines.

In principle, mixtures of more than one polyisocyanate and/or polyhydroxy compound can also be used, but the use of only one polyisocyanate is preferred. The molar ratio of NCO groups of the polyisocyanates to OH groups of the polyhydroxy compounds here is typically 25:1 to 1.5:1, preferably 20:1 to 1.5:1 and particularly preferably 15:1 to 1.5:1. The reaction generally takes place at temperatures of 20 to 140° C., preferably at 40 to 120° C. In principle, the reaction can be accelerated by using catalysts which are known per se from polyurethane chemistry, such as for example tin soaps, e.g. dibutyltin dilaurate, or tertiary amines, e.g. triethylamine or diazabicyclooctane (DABCO), but this method is not preferred. The addition of the components and optionally of a catalyst of the aforementioned type can, in principle, take place in any order. If the polyisocyanate is used in excess, it is preferred to separate this off after the reaction by extraction or distillation, preferably by thin-film distillation. The separation of the excess polyisocyanate is performed to the extent that less than 1 wt. %, preferably less than 0.5 wt. % and particularly preferably less than 0.2 wt. % of the polyisocyanate remains in the resulting polyisocyanate prepolymer.

As polyhydroxy compounds in B) it is possible to use all compounds known to the person skilled in the art which have an average OH functionality of at least 1.5. These can be for example low molecular weight diols (e.g. 1,2-ethanediol, 1,3- or 1,2-propanediol, 1,4-butanediol), triols (e.g. glycerol, trimethylolpropane) and tetraols (e.g. pentaerythritol), but also higher molecular weight polyhydroxy compounds such as polyether polyols, polyester polyols, polycarbonate polyols and polythioether polyols. However, those polyester polyols are preferred which have a hydroxyl number from 6 to 720 mg KOH/g, preferably from 28 to 480 mg KOH/g and particularly preferably from 40 to 240 mg KOH/g and an average functionality of 2 to 4, preferably 2 to 3.7 and particularly preferably from 2 to 3.6. These polyester polyols can be produced in a known manner by polycondensation of low molecular weight polycarboxylic acid derivatives, such as e.g. succinic acid, adipic acid, suberic acid, azelaic acid, sebacic acid, dodecanedioic acid, tetrahydrophthalic anhydride, hexahydrophthalic anhydride, tetrachlorophthalic anhydride, endomethylenetetrahydrophthalic anhydride, glutaric anhydride, maleic acid, maleic anhydride, fumaric acid, dimer fatty acid, trimer fatty acid, phthalic acid, phthalic anhydride, isophthalic acid, terephthalic acid, citric acid or trimellitic acid, with low molecular weight polyols, such as e.g. ethylene glycol, diethylene glycol, neopentyl glycol, hexanediol, butanediol, propylene glycol, glycerol, trimethylolpropane 1,4-hydroxymethylcyclohexane, 2-methyl-1,3-propanediol, 1,2,4-butanetriol, triethylene glycol, tetraethylene glycol, polyethylene glycol, dipropylene glycol, polypropylene glycol, dibutylene glycol and polybutylene glycol, or by ring-opening polymerisation of cyclic carboxylic acid esters, such as ε-caprolactone. Moreover, hydroxycarboxylic acid derivatives, such as e.g. lactic acid, cinnamic acid or ω-hydroxycaproic acid can also be polycondensed to form polyester polyols. It is also possible to use polyester polyols of oleochemical origin, however. These polyester polyols can be produced e.g. by complete ring opening of epoxidised triglycerides of an at least partially olefinically unsaturated fatty acid-containing fat mixture with one or more alcohols with 1 to 12 C atoms and subsequent partial transesterification of the triglyceride derivatives to form alkyl ester polyols with 1 to 12 C atoms in the alkyl radical.

The components of A) and B) are mixed together in a molar ratio of isocyanate groups to hydroxyl groups of 1:1 up to 1.8:1, preferably in a molar ratio of isocyanate groups:hydroxyl groups of 1:1 up to 1.6:1 and particularly preferably in a molar ratio of isocyanate groups:hydroxyl groups of 1.05:1 up to 1.5:1.

As additives C), the adhesive formulation may also contain, in addition to the above-mentioned components, additives known from adhesives technology as formulation auxiliaries. These additives are e.g. the conventional plasticisers, fillers, pigments, drying agents, light stabilisers, antioxidants, thixotropic agents, adhesion promoters and optionally other auxiliary substances and additives.

Examples of suitable fillers that may be mentioned are carbon black, precipitated silicas, pyrogenic silicas, mineral chalks and precipitated chalks.

Suitable plasticisers are e.g. phthalic acid ester, adipic acid ester, alkylsulfonic acid esters of phenol or phosphoric acid ester.

Examples of thixotropic agents that may be mentioned are pyrogenic silicas, polyamides, hydrogenated castor oil derivatives or polyvinyl chloride.

Suitable drying agents are in particular alkoxysilyl compounds, such as e.g. vinyltrimethoxysilane, methyltrimethoxysilane, methyltriethoxysilane, i-butyltrimethoxysilane, i-butyltriethoxysilane, octyltriethoxysilane, octyltrimethoxysilane, propyltriethoxysilane, propyltrimethoxysilane, hexadecyltrimethoxysilane, and inorganic substances such as e.g. calcium oxide (CaO) and isocyanate group-containing compounds such as e.g. tosyl isocyanate.

The known functional silanes are used as adhesion promoters, such as e.g. aminosilanes of the aforementioned type, but also N-aminoethyl-3-aminopropyltrimethoxysilane, N-amino ethyl-3-aminopropylmethyldimethoxysilane, N-aminoethyl-3-aminopropyltrimethoxysilane, 3-glycidoxypropyltrimethoxysilane, 3-glycidoxypropyltriethoxysilane, mercaptosilane, bis(3-triethoxysilylpropyl)amine, bis(3-trimethoxysilylpropyl)amine, oligoaminosilanes, 3-aminopropylmethyldiethoxysilane, 3-aminopropyltriethoxysilane, triaminofunctional propyltrimethoxysilane, N-(n-butyl)-3-aminopropyltrimethoxysilane, phenyltriethoxysilane, phenyltrimethoxysilane, polyether-functional trimethoxysilanes and 3-methacryloxypropyltrimethoxysilane.

The production of the adhesive formulation from the isocyanate group-containing component A) and the polyol or polyol mixture B) for the production of a film composite is known per se to the person skilled in the art from polyurethane chemistry. The additives C) may be added to the polyol or polyol formulation B) or to the isocyanate group-containing component A) or both. Preferably, the additives C) are added to the polyol or polyol formulation B).

In a preferred embodiment of the invention, the two components A) and B) of the adhesive formulation are mixed together immediately before the production of the film composite and introduced into the laminating machine or the applicator unit. In another preferred embodiment, the mixing of the components A) and B) may take place in the laminating machine itself immediately before or in the applicator unit. The adhesive formulation may be used here without solvents, or in a suitable solvent or solvent mixture. Suitable solvents are those which exhibit adequate solubility of the polyhydroxy component and the polyisocyanate component. Examples of these solvents are benzene, toluene, ethyl acetate, butyl acetate, propyl acetate, methyl ethyl ketone, methyl isobutyl ketone, 2-methoxypropyl acetate. Particularly preferred are ethyl acetate and methyl ethyl ketone. In the applicator unit, the so-called support film is coated with the adhesive formulation with an average dry application weight of 1 to 9 g/m² and, by bringing it into contact with a second film, it is laminated to form the resulting film composite. Optionally used solvents or solvent mixtures are removed completely in a drying tunnel or in another suitable device before the support film is brought into contact with the second film.

The adhesive formulation is preferably used for bonding plastics films, aluminium foils, other metal foils, plastics films with metal coatings and plastics films with metal oxide coatings.

The invention is explained by the following, non-restrictive examples.

### EXAMPLES

In the following examples, percentages refer to the weight. Unless otherwise specified, the viscosities were determined at a measuring temperature of 25° C. with the aid of the Viscotester VT 550 rotational viscometer from Thermo Haake, Karlsruhe, DE with the SV measuring cup and the SV DIN measuring device. The NCO content of the prepolymers or reaction mixtures was determined in accordance with DIN EN 1242.

The Following Abbreviations were Used:
OHN: Hydroxyl number [mg KOH/g]
AN: Acid number [mg KOH/g]
% NCO: NCO content in wt. % NCO groups
IA: Interlayer adhesion [N/15 mm] between the aluminium and the polyethylene layer in the following composite 12 μm polyethylene terephthalate/9 μm aluminium foil/60 μm polyethylene film

### Abbreviations of Reagents Used:

### Polyols:

P1: Polypropylene ether tetraol initiated with ethylenediamine, produced by KOH catalysis, OHN 470.
P2: Polypropylene ether diol initiated with 1,2-propylene glycol, produced by KOH catalysis, OHN 262.
P3: Polypropylene ether glycol, initiated with 1,2-propylene glycol, produced by KOH catalysis, OHN 112
P4: Polyester polyol as a reaction product of 14 parts by weight adipic acid, 39 parts by weight isophthalic acid, 7 parts by weight phthalic anhydride, 12 parts by weight trimethylolpropane and 42 parts by weight 1,6-hexanediol, OHN 141, AN≦3.
P5: Polyester polyol as a reaction product of 11.5 parts by weight adipic acid, 32.9 parts by weight isophthalic acid, 5.9 parts by weight phthalic anhydride, 13.4 parts by weight trimethylolpropane and 47.0 parts by weight 1,6-hexanediol, OHN 242, AN≦3.

### Polyisocyanates:

### NCO1:

Hexamethylene 1,6-diisocyanate (HDI) with a content of ≧99.5 wt. % and ≧49.7% NCO.

Aliphatic Prepolymer Containing Tert. Amino Groups According to the Invention:

1242 g P1 are added dropwise to 8757 g NCO1 at 100° C. and with continuous stirring within 2 hours. After complete conversion, the excess HDI is separated off at 130° C. and <1 mbar by distillation. A prepolymer is obtained with the following characteristics: viscosity (23° C., 40 s⁻¹) 139 Pas; 15.0% NCO, 0.18 wt. % free HDI.

Aliphatic Prepolymer A Free From Tert. Amino Groups not According to the Invention:

363 g P2 are added dropwise to 2137 g NCO1 at 100° C. and with continuous stirring within 2 hours. After complete conversion, the excess HDI is separated off at 130° C. and <1 mbar by distillation. A prepolymer is obtained with the following characteristics: viscosity (23° C., 40 s⁻¹) 1054 mPas; 10.91% NCO, 0.04 wt. % free HDI.

Aliphatic Prepolymer B Free from Tert. Amino Groups not According to the Invention:

2563 g P3 are added dropwise to 3936 g NCO1 at 100° C. and with continuous stirring within 2 hours. After complete conversion, the excess HDI is separated off at 130° C. and <1 mbar by distillation. A prepolymer is obtained with the following characteristics: viscosity (23° C., 40 s⁻¹) 1262 mPas; 6.49% NCO, 0.03 wt. % free HDI.

### Preparation of the Adhesive Formulation:

Since the mixture of the polyol component and the polyisocyanate component is by nature unsuitable for storage, this is produced immediately before production of the film composite.

The adhesive formulation is produced by intimate mixing of the polyol component and the polyisocyanate component. The mixture is produced with a 1.4× molar excess of isocyanate groups and is processed immediately.

### Production of the Film Composites Using the Adhesive Formulations Described in Table 1

The film composites are produced using a “Polytest 440” solvent-free laminating unit from Polytype in Freiburg, Switzerland.

The film composites are produced from a polyethylene terephthalate/aluminium precomposite and a polyethylene film. The aluminium side of the precomposite is coated with the adhesive formulation, bonded with the polyethylene film and then wound on to a roll core. The length of the film composite produced with the adhesive formulation is at least 20 m. The dry application quantity of the adhesive formulation is between 1.9 g and 2.8 g and the roll temperature of the applicator unit is 30-40° C.

TABLE 1
Batch quantities and testing of the adhesive formulations:

| | Adhesive formulation | Adhesive formulation | | |
|---|---|---|---|---|
| | according to the | not according to the | | |
| | invention | invention | | |
| Reagents in wt. % | 1 | a | b | c |
| Tertiary amino group-containing | 49.6 | | | |
| prepolymer according to the invention | | | | |
| Tertiary amino group-free prepolymer | | 69.9 | | |
| A not according to the invention | | | | |
| Tertiary amino group-free prepolymer | | | 69.9 | 69.9 |
| B not according to the invention | | | | |
| P4 | 50.4 | | 30.1 | 30.1 |
| P5 | | 30.1 | | |
| IA after x d at 23° C. | | | | |
| 1 | 2.3 | 0.0 | 0.0 | 1.3 |
| 2 | 4.3 | 0.2 | 0.0 | 1.4 |
| 3 | 4.7 | 2.6 | 0.7 | 1.2 |
| 7 | 4.3 | 2.0 | 1.2 | 1.5 |
| 14 | 3.6 | 1.7 | 1.2 | 1.2 |
| 28 | 3.6 | 1.6 | 1.5 | 1.5 |

1-13. (canceled)

14. Prepolymers based on aliphatic isocyanates, which contain tertiary amino groups bound to the prepolymer.

15. Prepolymers according to claim 14, characterised in that the tertiary amino groups are introduced into the prepolymer by the polyisocyanate component.

16. Prepolymers according to claim 14, characterised in that the tertiary amino groups are introduced into the prepolymer by the isocyanate-reactive component.

17. Prepolymers according to claim 14, characterised in that the aliphatic polyisocyanate used for their production has an NCO content of 11-51 wt. % and a nominal average functionality of 2 to 3.8.

18. Preparations containing prepolymers according to claim 14.

19. Preparations according to claim 18, characterised in that they are adhesives.

20. Use of preparations according to claim 18 in the production of adhesive and plaster systems for wound closure and care.

21. Substrates coated with preparations according to claim 17.

22. Substrates according to claim 21, characterised in that they are packaging materials.

23. Use of adhesives according to claim 19 for the production of film composites.

24. Use according to claim 23, characterised in that these are composite films.

25. Use according to claim 23, characterised in that these are films for food packaging.

26. Foods at least partly packed with substrates according to claim 21.
